(19) United States
(12) Patent Application Publication
(10) Pub. No.: US 2020/0249227 A1
(43) Pub. Date: Aug. 6, 2020

(54) METHOD FOR DETECTING AND QUANTIFYING VITAMINS AND THYROID ANALYTES FROM A URINE SAMPLE

(71) Applicant: Alcala Pharmaceutical, Inc., San Diego, CA (US)

(72) Inventors: David J. Smith, San Diego, CA (US); Christian Tagwerker, Oceanside, CA (US); Irfan Baig, San Diego, CA (US)

(21) Appl. No.: 16/773,644

(22) Filed: Jan. 27, 2020

Related U.S. Application Data
(60) Provisional application No. 62/799,550, filed on Jan. 31, 2019.

(51) Int. Cl. G01N 33/543; G01N 33/78; G01N 33/82

(52) U.S. Cl. CPC G01N 33/54306; G01N 33/82; G01N 33/78

(57) ABSTRACT

A method for analyzing a urine sample includes binding a plurality of analytes from a urine sample to an adsorbent; and analyzing the plurality of analytes using a molecular analyzer to detect evidence of at least one water soluble vitamin and at least one fat soluble vitamin in the urine sample. The method can also include analyzing the urine sample using the molecular analyzer to detect a thyroid analyte selected from the group consisting of 3-5-Diiodo-L-Thyronine (LT2), 3-3′-5′-L-Thyroxine (LT3) and L-Thyroxine (LT4). Prior to analyzing the analytes, the method can include one or more of: washing the solid phase extraction column with a methanol solution after the step of binding; eluting the plurality of analytes from the adsorbent with at least one of (i) a mixture of isopropanol and methanol, and (ii) a mixture of ethyl acetate and methanol, to form an eluate; evaporating the eluate; and reconstituting the evaporated eluate with a mixture of methanol and water.

### BACKGROUND

Vitamins are a group of organic compounds that are essential for normal growth and nutrition and are required in small quantities in the diet because they cannot be synthesized by the body. Some of these vitamins are fat-soluble (hydrophobic) and some are water-soluble (hydrophilic). For example, vitamins such as Vitamin A, D, E and K dissolve in fats. Other vitamin compounds, such as Vitamin B1, B2, B3, B5, B6, B9, B12, C and Folic Acid, are water soluble.

Vitamins play a key role in our body but monitoring vitamin levels becomes important if one is suffering from a serious disease or taking prescription medication that can directly affect one or more vitamin levels. Generally, vitamin levels are analyzed in blood (serum/plasma) in specimens collected by venipuncture. Some patients are difficult to obtain a venipuncture blood sample from due to their existing condition. On the other hand, acquiring biological samples by other methods can be more convenient, can cause little or no pain, and can generally involve relatively non-invasive processes for obtaining such samples from patients.

Additionally, determining thyroid hormone levels in patients is important to assess thyroid dysfunction, particularly low-grade hypothyroidism that may otherwise go undetected using conventional testing methods.

Liquid chromatography-mass spectrometry (LC-MS) and liquid chromatography tandem mass spectrometry (LC-MS/MS) are analytical chemistry techniques that combine the physical separation capabilities of liquid chromatography (or HPLC) with the mass analysis capabilities of mass spectrometry (MS). These systems are popular in chemical analysis because the individual capabilities of each technique are enhanced synergistically. While liquid chromatography separates mixtures with multiple components, mass spectrometry provides structural identity of the individual components with high molecular specificity and detection sensitivity.

In recent years, liquid chromatography tandem mass spectrometry (LC-MS/MS) has emerged as an innovative analytical technology applicable to some analyses in the endocrinology laboratory. In tandem MS (MS/MS) two quadrupole mass filters are combined and target analyte molecules from the first quadrupole are submitted to a controlled fragmentation in a collision cell. The entirety of ions is transferred into the first quadrupole mass filter. Here, the mass-to-charge ratio (m/z) of the intact ionized target analyte is selected, and all other ion species are filtered out. The selected ions sharing identical m/z are continuously transferred into the collision cell. Ions selected by the first quadrupole fragment into characteristic product ions. For various analytes, several characteristic, thermodynamically favored product ions are generated. These fragment ions are guided to the second quadrupole. The radiofrequency settings of the second analytical quadrupole are adjusted in a way that only one selected fragment ion will pass, while all other fragment ion species are filtered out. Thus, one defined “daughter ion” from one defined “parent ion” finally reaches an ion detector, and can then be identified and/or quantified.

The presence of vitamins and/or their metabolites, as well as thyroid analytes, in urine as well as certain other types of biological samples is well-known. However, conventional detection and/or quantification methods either focus on one specific vitamin with its possible deficiency marker, on a select class of vitamins, such as either fat-soluble or water-soluble vitamins, or on explicit thyroid analytes such as 3-3′-5′-L-Thyroxine (LT3) or L-Thyroxine (LT4). In clinical practice, the treating physician should be given as broad and accurate an assessment of vitamins and thyroid hormone levels that are present in normal quantities, absent, or deficient in the patient's system as possible. A procedure is needed to concurrently determine the concentration and/or presence of one or more water-soluble and one or more fat-soluble vitamins, their metabolites, their deficiency markers and/or thyroid hormones in easier-to-access urine or other biological samples.

### SUMMARY

The present invention is directed toward a method for analyzing a urine sample. In certain embodiments, the method includes the steps of binding a plurality of analytes from the urine sample to an adsorbent; and analyzing the plurality of analytes using a molecular analyzer to detect evidence of at least one water soluble vitamin and at least one fat soluble vitamin in the urine sample.

In various embodiments, the method can also include the step of analyzing at least a portion of the urine sample using the molecular analyzer to detect evidence of a thyroid analyte selected from the group consisting of 3-5-Diiodo-L-Thyronine (LT2), 3-3′-5′-L-Thyroxine (LT3) and L-Thyroxine (LT4) in the urine sample substantially concurrently with analyzing the plurality of analytes.

In other embodiments, the method can also include the step of analyzing at least a portion of the urine sample using the molecular analyzer to detect evidence of at least two thyroid analytes selected from the group consisting of 3-5-Diiodo-L-Thyronine (LT2), 3-3′-5′-L-Thyroxine (LT3) and L-Thyroxine (LT4) in the urine sample substantially concurrently with analyzing the plurality of analytes.

In some embodiments, the method can also include the step of analyzing at least a portion of the urine sample using the molecular analyzer to detect evidence of 3-5-Diiodo-L-Thyronine (LT2) in the urine sample substantially concurrently with analyzing the plurality of analytes.

In various embodiments, the method can also include the step of quantifying an amount of at least one water soluble vitamin in the urine sample using the molecular analyzer.

In certain embodiments, the method can also include the step of quantifying an amount of at least one fat soluble vitamin in the urine sample using the molecular analyzer.

In some embodiments, the method can also include the step of quantifying an amount of at least one of 3-5-Diiodo-L-Thyronine (LT2), 3-3′-5′-L-Thyroxine (LT3) and L-Thyroxine (LT4) in the urine sample using the molecular analyzer simultaneously with the step of analyzing the one or more analytes.

In various embodiment, the step of binding can include applying the urine sample and a β-glucuronidase buffer mix to a solid phase extraction column.

In certain embodiments, the solid phase extraction can include a hydrophilic and/or lipophilic solid phase extraction.

In some embodiments, the hydrophilic and/or lipophilic solid phase extraction can include a copolymeric resin.

In various embodiments, the method can also include the step of washing the solid phase extraction column with a methanol solution after the step of binding.

In certain applications, the method can also include the step of eluting the plurality of analytes from the adsorbent with at least one of (i) a mixture of isopropanol and methanol, and (ii) a mixture of ethyl acetate and methanol, so that an eluate is formed.

In some embodiments, the method can also include the steps of (i) evaporating the eluate, and (ii) reconstituting the evaporated eluate with a mixture of methanol and water.

In another embodiment, the present invention is directed toward a method for analyzing a urine sample. In various embodiments, the method can include the steps of eluting a plurality of analytes from the urine sample that are bound to an adsorbent with at least one of (i) a mixture of isopropanol and methanol, and (ii) a mixture of ethyl acetate and methanol, so that an eluate is formed; and analyzing the plurality of analytes using a molecular analyzer to detect evidence of at least one water soluble vitamin and at least one fat soluble vitamin in the urine sample.

In some such embodiments, the method can also include the step of washing the adsorbent with a methanol solution before the step of eluting.

Additionally, or in the alternative, the method can include the steps of (i) evaporating the eluate, and (ii) reconstituting the evaporated eluate with a mixture of methanol and water.

In certain embodiments, the method can also include the step of analyzing at least a portion of the urine sample using the molecular analyzer to detect evidence of a thyroid analyte selected from the group consisting of 3-5-Diiodo-L-Thyronine (LT2), 3-3′-5′-L-Thyroxine (LT3) and L-Thyroxine (LT4) in the urine sample substantially concurrently with analyzing the plurality of analytes.

The present invention is also directed toward a method for analyzing a urine sample, having the steps of washing with a methanol solution at least a portion of the urine sample that is bound to an adsorbent; and analyzing a plurality of analytes using a molecular analyzer to detect evidence of at least one water soluble vitamin and at least one fat soluble vitamin in the urine sample.

In various embodiments, the methanol solution can contain between 1-99% methanol by volume.

In certain embodiments, the method can also include the step of analyzing at least a portion of the urine sample using the molecular analyzer to detect evidence of a thyroid analyte selected from the group consisting of 3-5-Diiodo-L-Thyronine (LT2), 3-3′-5′-L-Thyroxine (LT3) and L-Thyroxine (LT4) in the urine sample substantially concurrently with analyzing the plurality of analytes.

The present invention is also directed toward a method for analyzing a urine sample having the steps of binding a plurality of analytes from the urine sample to an adsorbent; washing with a methanol solution the plurality of analytes that are bound to the adsorbent; eluting the plurality of analytes from the urine sample that are bound to the adsorbent with at least one of (i) a mixture of isopropanol and methanol, and (ii) a mixture of ethyl acetate and methanol, so that an eluate is formed; evaporating the eluate; reconstituting the evaporated eluate with a mixture of methanol and water; and analyzing the plurality of analytes using a molecular analyzer to detect evidence of at least one water soluble vitamin and at least one fat soluble vitamin in the urine sample.

In various embodiments, the method can also include the step of analyzing at least a portion of the urine sample using the molecular analyzer to detect evidence of a thyroid analyte selected from the group consisting of 3-5-Diiodo-L-Thyronine (LT2), 3-3′-5′-L-Thyroxine (LT3) and L-Thyroxine (LT4) in the urine sample substantially concurrently with analyzing the plurality of analytes.

### BRIEF DESCRIPTION OF THE DRAWING

The novel features of this invention, as well as the invention itself, both as to its structure and its operation, will be best understood from the accompanying figure, taken in conjunction with the accompanying description, in which similar reference characters refer to similar parts, and in which:

FIG. 1 is a flow chart showing one embodiment of a method for detecting and quantifying vitamins and/or thyroid presence from a urine sample.

### DESCRIPTION

Embodiments of the present invention are described herein in the context of a method for detecting and quantifying vitamins and/or thyroid activity from a biological sample such as a urine sample. Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Further, it is understood that although the description herein regarding biological samples is somewhat directed toward analysis of urine samples, such is provided for the sake of clarity and simplicity. It is recognized that other suitable biological samples can equally and effectively be analyzed utilizing one or more of the methods disclosed herein. These other biological samples can include, without limitation, whole blood, blood plasma, serum, dried blood, saliva (oral fluids), cerebrospinal fluid (CSF), tears, sweat, semen, feces, etc. The types of dried blood samples can vary, and can include, without limitation, samples obtained via Neoteryx® blood collection devices, as one non-exclusive example.

As used herein, for the sake of simplicity, the term “vitamin”, such as in “water-soluble vitamin” and “fat-soluble vitamin” is intended to include vitamins, vitamin metabolites and vitamin deficiency markers. A non-exclusive listing of 34 vitamins, vitamin metabolites and vitamin deficiency markers (also sometimes referred to herein as “analytes”) includes, but is not limited to:

Retinoic acid (Vitamin A)

4-KRME (4-Keto-Retinoic Acid-methyl-ester)

Vitamin D2 (CalciferolErgocalciferol)

25-Hydroxyvitamin D2 (25-hydroxyergocalciferoVErcalcidiol)

1-25-Dihydroxy-Vitamin-D2 (Ercalcitriol)

Vitamin D3 (Cholecalciferol)

25-Hydroxyvitamin D3 (Calcidiol)

1-25-Dihydroxy-Vit-D3 (Calcitriol)

L-Thyroxine

3-3-Diiodo-L-thyronine (LT2)

3-3′-5′-L-Thyroxine (LT3)

Alpha-CEHC (Vitamin E metabolite)

Gamma-CEHC (Vitamin E metabolite)

Menadione (Vitamin K3)

Menaquinone-4

Riboflavin (Vitamin B2)

Pyridoxine (Vitamin B6)

Pyridoxal (Vitamin B6)

Xanthurenic Acid (Vitamin B6 deficiency marker)

Folic Acid (Vitamin B9)

FA apABG (Folic Acid metabolite)

4-Amino-benzoic Acid (pABA)

10-FormylFolicAcid

Cyanocobalamin (Vitamin B12)

Methylmalonate (Vitamin 812 deficiency marker)

Pantothenic-Acid (Vitamin B5)

Ascorbic Acid (Vitamin C)

Nicotinamide-Niacin (Vitamin B3)

Thiamin (Vitamin B1)

D-Biotin (Vitamin B9)

250HVD3-PTAD (**) (**) designates derivatized forms of Vitamin D metabolites.

250HVD2-PTAD

1-25DiOHVD3-PTAD

24R-25DiOHVD3-PTAD

The analytes described herein can also include various thyroid hormones or markers, such as 3-5-Diiodo-L-Thyronine (LT2), 3-3′-5′-L-Thyroxine (LT3) and L-Thyroxine (LT4).

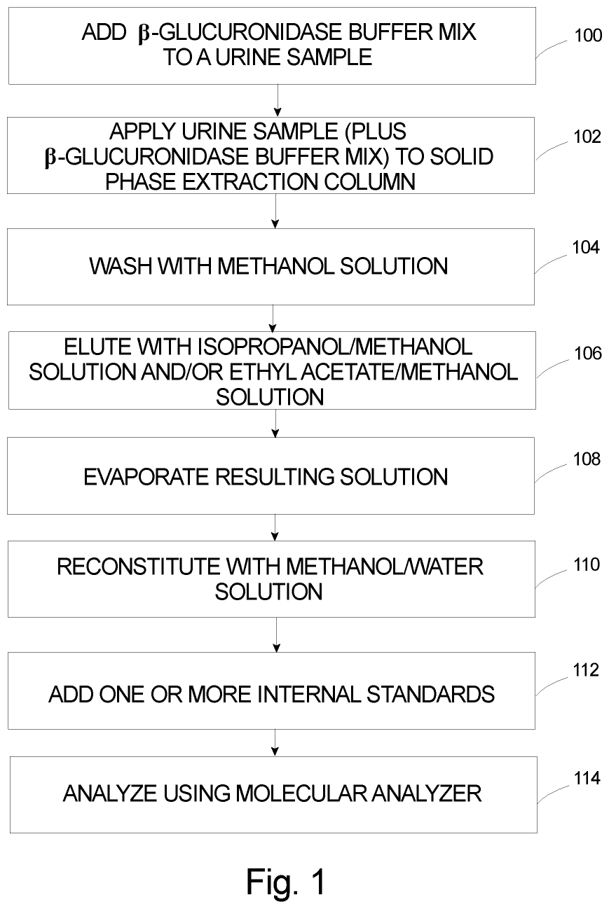

FIG. 1 is a flow chart showing one embodiment of a method for detecting and quantifying vitamins and/or thyroid activity from a biological sample such as a urine sample. The biological sample can come from patients under the care of a treating physician from clinics and clinical institutions or from private parties. The methods described herein include novel robust, highly specific and reliable, e.g. free of interference, chromatographic separation methods. It is recognized that in certain embodiments, various steps illustrated and described with respect to FIG. 1 can be omitted without deviating from the scope of the disclosure herein. It is further recognized that in some embodiments, additional steps can be included in the method shown in FIG. 1 that are not illustrated and described. Additionally, it is understood that although urine is the biological sample that is specifically described relative to the description of FIG. 1, any other suitable biological sample could equally be utilized. Thus, as used herein, “urine sample” is but one example of the “biological sample”. In certain non-exclusive embodiments, the method for detecting and quantifying vitamins and/or thyroid activity from the urine sample can include one or more of the following steps.

At step 100, a β-glucuronidase buffer mix can be added to a urine sample. The specific β-glucuronidase buffer mix that can be used can vary. In certain embodiments, the β-glucuronidase buffer mix can include a sodium acetate (C₂H₃NaO₂) buffer solution. In one embodiment, the β-glucuronidase buffer mix can include a 100 mM sodium acetate buffer solution having an approximate pH 4.8. The buffer solution can then be added to a β-glucuronidase enzyme. In one embodiment, buffer solution can then be added to a β-glucuronidase enzyme such as BG100 β-glucuronidase to achieve the desired concentration. In some embodiments, the desired concentration can range from 10-1,000,000 Units/mL. In one embodiment, the desired concentration can be at least approximately 100,000 Units/mL. Alternatively, the β-glucuronidase buffer mix can be prepared using other suitable methods and/or compounds in order to achieve the desired mixture and/or concentration.

In one embodiment, the β-glucuronidase buffer mix can be added to the urine sample so that the concentration of β-glucuronidase buffer mix is between approximately 10-100,000 Units/mL. In non-exclusive alternative embodiments, the β-glucuronidase buffer mix can be added to the urine sample so that the concentration of β-glucuronidase buffer mix is between approximately 100-10,000 Units/mL, between approximately 500-5,000 Units/mL or between approximately 1,000-2,000 Units/mL. Still alternatively, the β-glucuronidase buffer mix can be added to the urine sample so that the concentration of β-glucuronidase buffer mix is outside of the foregoing ranges.

At step 102, the urine sample (and the β-glucuronidase buffer mix) are applied to a solid phase extraction (SPE) column. The specific type of SPE can vary. In one embodiment, a hydrophilic and/or lipophilic SPE can be used which includes a copolymeric resin (also sometimes referred to herein as an “adsorbent”). In one such non-exclusive embodiment, the hydrophilic and/or lipophilic SPE can be used which includes both a hydrophilic and a reverse-phase (lipophilic) resin. Alternatively, another suitable type of SPE can be used. During step 102, one or more analytes from the urine sample, if present, are bound to an adsorbent in the SPE column. In various embodiments, the analytes can include one or more vitamins, which can include water-soluble or fat-soluble vitamins, their metabolites and/or their deficiency markers.

Further, or in the alternative, the analytes can include one or more of certain thyroid hormones, including 3-5-Diiodo-L-Thyronine (LT2), 3-3′-5′-L-Thyroxine (LT3) and/or L-Thyroxine (LT4).

At step 104, the column is washed with a methanol solution. In one embodiment, the methanol solution can be within the range of approximately 1-99% methanol by volume. In non-exclusive alternative embodiments, the methanol solution can be within the range of approximately 1-50% methanol by volume, 1-20% methanol by volume, 1-10% methanol by volume or 1-5% methanol by volume. Still alternatively, the methanol solution can be approximately 5% methanol by volume. In certain embodiments, the methanol solution includes only methanol and water. In one embodiment, the methanol solution includes only high-performance liquid chromatography grade methanol and high-performance liquid chromatography grade water.

In various embodiments, the ratio of the volume of the biological sample used to the volume of the methanol solution can be less than approximately 500:1. In non-exclusive alternative embodiments, the ratio of a volume of the biological sample to a volume of the methanol solution can be less than approximately 100:1 or 50:1. Still alternatively, the ratio of the volume of the biological sample to the volume of the methanol solution can be greater than approximately 5:1, 2:1 or 1:1. In yet another embodiment, the ratio of the volume of the biological sample to the volume of the methanol solution can be approximately 10:1.

At step 106, the adsorbent is eluted with an isopropanol/methanol solution and/or an ethyl acetate/methanol solution to form an eluate which contains one or more analytes. In one embodiment, the ratio of the mixture of isopropanol to methanol is at least approximately 1:10. In non-exclusive alternative embodiments, the ratio of the mixture of isopropanol and methanol is at least approximately 1:5 or 1:2. Alternatively, the ratio of the mixture of isopropanol and methanol is less than approximately 10:1, 5:1 or 2:1. In yet another embodiment, the ratio of the mixture of isopropanol and methanol is approximately 1:1.

In certain embodiments, the ratio of the mixture of ethyl acetate and methanol is approximately 1:5. In non-exclusive alternative embodiments, the wherein a ratio of the mixture of ethyl acetate and methanol is greater than approximately 1:50, 1:20 or 1:10. Still alternatively, the ratio of the mixture of ethyl acetate and methanol is less than approximately 1:2, 1:1, 2:1 or 5:1.

At step 108, the eluate from step 106 is evaporated to form an evaporated eluate. The evaporation can occur at any suitable temperature. In one embodiment, the evaporation can occur at approximately 40 degrees C. At this point, the evaporated eluate can undergo 4-Phenyl-1,2,4-Triazoline-3,5-Dione (PTAD) derivatization, which can aid in boosting analytical sensitivity in one or more of the steps that follow.

At step 110, the evaporated eluate can be reconstituted. In certain embodiments, the evaporated eluate can be reconstituted with a methanol/water solution. In various embodiments, the ratio of methanol to water in the methanol/water solution can be less than approximately 10:1, 5:1 or 2:1. In non-exclusive alternative embodiments, the ratio of methanol to water in the methanol/water solution can be greater than 1:10, 1:5 or 1:2. Still alternatively, the ratio of methanol to water in the methanol/water solution can be approximately 1:1.

At step 112, one or more Internal Standards can be added to the reconstituted eluate. The amount of, and the specific Internal Standards that can be added can vary. In one embodiment, at least one of the following Internal Standards can be added: Candesartan-d5, Imipramine-d3, Xanthurenic Acid-d4, 3,4-Methylenedioxypyrovalerone-d8 Hydrochloride (MDPV-D8), Haloperidol-d4, Naltrexone-d3 and Creatinine-d3. Alternatively, at least two of the foregoing Internal Standards can be added. Still alternatively, at least three of the foregoing Internal Standards can be added. In another embodiment, the Internal Standard(s) that are added can include any other suitable Internal Standard(s).

At step 114, the resultant mixture from step 112 can then be analyzed using a molecular analyzer (also sometimes referred to herein simply as an “analyzer”). As used herein, the terms “molecular analyzer” or “analyzer” can include LC-MS, LC-MS/MS, GC-MS, HPLC, HPLC-UV-Vis, MALDI-TOF Mass Spectrometry, Fourier Transform Mass Spectrometry, immunoassay-based analyzers, etc. as non-exclusive examples, or any other suitable method of analyzing the resultant mixture known to those skilled in the art. The analyzer is used to detect the analytes in the urine sample. In various embodiments, due to the methods described herein, the analyzer can concurrently yield evidence of at least one water soluble vitamin and at least one fat soluble vitamin in the urine sample. Additionally, or in the alternative, the analyzer can concurrently yield evidence of one or more of 3-5-Diiodo-L-Thyronine (LT2), 3-3′-5′-L-Thyroxine (LT3) and L-Thyroxine (LT4) in the urine sample. In certain embodiments, the analyzer can yield evidence of any single analyte, any combination of analytes, or all of the analytes listed and/or described herein during a single molecular analyzer event. Stated another way, one or more such analyses of different analytes and/or 3-5-Diiodo-L-Thyronine (LT2), 3-3′-5′-L-Thyroxine (LT3) and L-Thyroxine (LT4) can occur substantially simultaneously with one another. The analytes can be determined by any suitable method, including, but not limited to, consulting a lookup table, printouts from the analyzer process(es), appropriate software, etc.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

As used herein, the recitation of numerical ranges by endpoints shall include all numbers subsumed within that range, inclusive (e.g., 2 to 8 includes 2, 2.1, 2.8, 5.3, 7, 8, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the method for detecting and quantifying vitamins and/or thyroid activity from biological samples have been illustrated and described herein, one or more features of any one embodiment can be combined with one or more features of one or more of the other embodiments, provided that such combination satisfies the intent of the present invention.

While a number of exemplary aspects and embodiments of the method for detecting and quantifying vitamins and/or thyroid activity from biological samples have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

What is claimed is:

1. A method for analyzing a urine sample, the method comprising the steps of:
binding a plurality of analytes from the urine sample to an adsorbent; and
analyzing the plurality of analytes using a molecular analyzer to detect evidence of at least one water soluble vitamin and at least one fat soluble vitamin in the urine sample.

2. The method of claim 1 further comprising the step of analyzing at least a portion of the urine sample using the molecular analyzer to detect evidence of a thyroid analyte selected from the group consisting of 3-5-Diiodo-L-Thyronine (LT2), 3-3′-5′-L-Thyroxine (LT3) and L-Thyroxine (LT4) in the urine sample substantially concurrently with analyzing the plurality of analytes.

3. The method of claim 1 further comprising the step of analyzing at least a portion of the urine sample using the molecular analyzer to detect evidence of at least two thyroid analytes selected from the group consisting of 3-5-Diiodo-L-Thyronine (LT2), 3-3′-5′-L-Thyroxine (LT3) and L-Thyroxine (LT4) in the urine sample substantially concurrently with analyzing the plurality of analytes.

4. The method of claim 1 further comprising the step of analyzing at least a portion of the urine sample using the molecular analyzer to detect evidence of 3-5-Diiodo-L-Thyronine (LT2) in the urine sample substantially concurrently with analyzing the plurality of analytes.

5. The method of claim 1 further comprising the step of quantifying an amount of at least one water soluble vitamin in the urine sample using the molecular analyzer.

6. The method of claim 1 further comprising the step of quantifying an amount of at least one fat soluble vitamin in the urine sample using the molecular analyzer.

7. The method of claim 1 further comprising the step of quantifying an amount of at least one of 3-5-Diiodo-L-Thyronine (LT2), 3-3′-5′-L-Thyroxine (LT3) and L-Thyroxine (LT4) in the urine sample using the molecular analyzer simultaneously with the step of analyzing the one or more analytes.

8. The method of claim 1 wherein the step of binding includes applying the urine sample and a β-glucuronidase buffer mix to a solid phase extraction column.

9. The method of claim 8 wherein the solid phase extraction includes a hydrophilic and/or lipophilic solid phase extraction.

10. The method of claim 9 wherein the hydrophilic and/or lipophilic solid phase extraction includes a copolymeric resin.

11. The method of claim 8 further comprising the step of washing the solid phase extraction column with a methanol solution after the step of binding.

12. The method of claim 1 further comprising the step of eluting the plurality of analytes from the adsorbent with at least one of (i) a mixture of isopropanol and methanol, and (ii) a mixture of ethyl acetate and methanol, so that an eluate is formed.

13. The method of claim 12 further comprising the steps of (i) evaporating the eluate, and (ii) reconstituting the evaporated eluate with a mixture of methanol and water.

14. A method for analyzing a urine sample, the method comprising the steps of:
eluting a plurality of analytes from the urine sample that are bound to an adsorbent with at least one of (i) a mixture of isopropanol and methanol, and (ii) a mixture of ethyl acetate and methanol, so that an eluate is formed; and
analyzing the plurality of analytes using a molecular analyzer to detect evidence of at least one water soluble vitamin and at least one fat soluble vitamin in the urine sample.

15. The method of claim 14 further comprising the step of washing the adsorbent with a methanol solution before the step of eluting.

16. The method of claim 14 further comprising the steps of (i) evaporating the eluate, and (ii) reconstituting the evaporated eluate with a mixture of methanol and water.

17. The method of claim 14 further comprising the step of analyzing at least a portion of the urine sample using the molecular analyzer to detect evidence of a thyroid analyte selected from the group consisting of 3-5-Diiodo-L-Thyronine (LT2), 3-3′-5′-L-Thyroxine (LT3) and L-Thyroxine (LT4) in the urine sample substantially concurrently with analyzing the plurality of analytes.

18. A method for analyzing a urine sample, the method comprising the steps of:
washing with a methanol solution at least a portion of the urine sample that is bound to an adsorbent; and
analyzing a plurality of analytes using a molecular analyzer to detect evidence of at least one water soluble vitamin and at least one fat soluble vitamin in the urine sample.

19. The method of claim 18 wherein the methanol solution contains between 1-99% methanol by volume.

20. The method of claim 18 further comprising the step of analyzing at least a portion of the urine sample using the molecular analyzer to detect evidence of a thyroid analyte selected from the group consisting of 3-5-Diiodo-L-Thyronine (LT2), 3-3′-5′-L-Thyroxine (LT3) and L-Thyroxine (LT4) in the urine sample substantially concurrently with analyzing the plurality of analytes.

21. A method for analyzing a urine sample, the method comprising the steps of:
binding a plurality of analytes from the urine sample to an adsorbent;
washing with a methanol solution the plurality of analytes that are bound to the adsorbent;
eluting the plurality of analytes from the urine sample that are bound to the adsorbent with at least one of (i) a mixture of isopropanol and methanol, and (ii) a mixture of ethyl acetate and methanol, so that an eluate is formed;
evaporating the eluate;
reconstituting the evaporated eluate with a mixture of methanol and water; and
analyzing the plurality of analytes using a molecular analyzer to detect evidence of at least one water soluble vitamin and at least one fat soluble vitamin in the urine sample.

22. The method of claim 21 further comprising the step of analyzing at least a portion of the urine sample using the molecular analyzer to detect evidence of a thyroid analyte selected from the group consisting of 3-5-Diiodo-L-Thyronine (LT2), 3-3′-5′-L-Thyroxine (LT3) and L-Thyroxine (LT4) in the urine sample substantially concurrently with analyzing the plurality of analytes.
